(19) United States
(12) Patent Application Publication
(10) Pub. No.: US 2026/0103757 A1
(43) Pub. Date: Apr. 16, 2026

(54) USING THE CONTENTS OF SALIVARY EXTRACELLULAR VESICLES AS BIOMARKERS FOR ALZHEIMER'S DISEASE AND ALZHEIMER'S DISEASE-RELATED DISEASES

(71) Applicants: BROWN UNIVERSITY, Providence, RI (US); RHODE ISLAND HOSPITAL, Providence, RI (US)

(72) Inventors: Jill Kreiling, Warren, RI (US); Peter Quesenberry, Providence, RI (US); Zhijin Wu, Barrington, RI (US)

(21) Appl. No.: 19/359,020

(22) Filed: Oct. 15, 2025

Related U.S. Application Data
(60) Provisional application No. 63/707,500, filed on Oct. 15, 2024.

(51) Int. Cl. C12Q 1/6883

(52) U.S. Cl. CPC C12Q 1/6883; C12Q 2600/158

(57) ABSTRACT

A method of identifying subjects having an increased risk of having or developing Alzheimer's disease (AD) or an Alzheimer's disease-related disorder (ARDR) is described. The method includes the steps of obtaining a saliva sample from the subject; isolating extracellular vesicles from the saliva sample; determining the level of a protein or a category of RNA present in the extracellular vesicles; comparing the level of the protein or category of RNA to a control level; and determining that the subject has an increased risk of having or developing AD or an ARDR if the protein or category of RNA level is altered relative to that of the control level. A method of evaluating treatment of AD or an ARDR in a subject receiving treatment for AD or an ARDR is also described.

### CROSS-REFERENCE TO RELATED APPLICATIONS

This application claims the benefit of U.S. Provisional Application Ser. No. 63/707,500, filed 15 Oct. 2024, entitled “METHODS OF USING SALIVARY EXTRACELLULAR VESICLES AS BIOMARKERS”. The entirety of these applications is incorporated by reference for all purposes.

### GOVERNMENT FUNDING

The present invention was made with government support under Grant No. R01AG074284 awarded by the National Institutes of Health. The US government has certain rights in this invention.

### SEQUENCE LISTING

The instant application contains a Sequence Listing which has been submitted electronically in XML format and is hereby incorporated by reference in its entirety. Said XML copy, created on Oct. 15, 2025, is named BU-030682W0-ORD.st.26 and is 5,549 bytes in size.

### BACKGROUND

Human saliva contains numerous factors such as proteins, DNA, and RNA, many of which are contained in extracellular vesicles (EVs). The composition of saliva can be affected by the health status of the individual, and the ease of collection has led to saliva being investigated for potential biomarkers of disease. Gardner et al., Metabolites, 10(2):47 (2020). Saliva has been shown to contain many of the same metabolites that are found in blood, likely due to the proximity of the salivary glands to blood vessels allowing for exchange of components between the blood and saliva. Yan et al., Proteomics Clin Appl., 3(1):116-34 (2009). Also, salivary glands are innervated by the autonomic nervous system, which is influenced by CNS activity. CNS conditions that impact autonomic function can lead to changes in salivary composition which may reflect neurological alterations in the CNS. Naumova et al., Sci Rep., 4:4884 (2014). In addition, there is high correlation between metabolites found in cerebrospinal fluid and saliva, making analysis of saliva an attractive, non-invasive screening mechanism for changes in the central nervous system. Reuster et al., Psychopharmacology (Berl), 162(4):415-8 (2002).

EVs are small membrane bound particles that are released by most cell types, including cell types found in the central nervous system (Faure et al., Mol Cell Neurosci., 31(4):642-8 (2006). Originally thought to be cellular junk, as a way of the cell taking out the trash, they are now recognized as potent mediators of intercellular communication. There are 3 types of EVs that differ by their size and method of production. Exosomes, the smallest of the EVs, are produced by the endosomal pathway and range in size from 30-100 nm. Microvesicles range in size from 100 nm-1 μm and apoptotic bodies are typically between 1-4 μm, and both are produced by direct shedding from the plasma membrane. Once released from the cell of origin EVs can target cells in the immediate vicinity or can enter the bloodstream and affect cells that are far from the cell of origin. EV cargo includes mRNA, miRNA, other ncRNAs, DNA, lipids and protein in a cell type specific manner that is believed to closely mirror the cytoplasmic content of the cell of origin. Yokoi et al., Sci Adv, 5(11):eaax8849 (2019). Once an EV reaches its target cell, it can release its cargo into the cytoplasm of that cell. RNA released into a target cell is translated using the target cell translation machinery, since EVs do not contain ribosomal RNAs. EVs can cross epithelial barriers by transcytosis allowing them to cross from the blood stream into the saliva. Guo and Jiang, Acta Pharm Sin B., 10(6):979-86 (2020). This mechanism also allows EVs to cross the blood brain barrier, allowing for accumulation of EVs derived from the central nervous system in blood, which can ultimately lead to their accumulation in saliva.

It was recently shown that the RNA content of salivary EVs (salEVs) can distinguish people who have a traumatic brain injury from those who have not. Cheng et al., Neural Regen Res., 15(4):676-81 (2020). It has also been shown that Alzheimer's disease (AD) associated proteins can be detected in saliva. Sabbagh et al., Neurol., 18(1):155 (2018).

Alzheimer's and related dementias are increasing, with the number of Americans with the condition projected to reach 14 million by 2060, driven by the aging population and factors like hypertension, diabetes, obesity, and a lack of exercise. Accordingly, there remains a need for methods of diagnosing subjects having or having an increased risk of developing these conditions.

### SUMMARY OF THE INVENTION

A major drawback to using extracellular vesicles (EV) isolated from blood is that most cell types produce EVs that enter the bloodstream, diluting the EVs that originate specifically from the brain. Saliva contains less of these contaminating EVs and therefore has proportionately more EVs produced in the central nervous system due to direct innervation of the salivary gland by cranial nerves VII and XI that originate in the brain. In addition, hemolysis can contaminate samples, skewing the results. Saliva, on the other hand, is free from blood, eliminating this issue. While blood is considered to be easily accessible, saliva is even more easily accessible and doesn't require specialized training or equipment to collect.

The inventors characterized the RNA cargo of extracellular vesicles (EVs) isolated from saliva (salEVs) from individuals over the age of 65 with normal cognition. Human saliva contains numerous factors, including DNA, RNA, and protein, that may reflect the health status of the individual. Many of these factors are contained within EVs. The contents of EVs are thought to mirror the cytoplasm of the cell of origin, providing insight into the health of the cell. The salEV RNA content was analyzed by RNA-seq and NanoString miRNA analysis. It was found that approximately 48.4% of the reads mapped to the human genome, with the remainder mapping to prokaryotic genomes. The transcripts included protein-coding RNA, long non-coding RNA, retrotransposons, and miRNAs. A significant number of the protein-coding transcripts were associated with pathways involved in neurodegenerative conditions. In addition, there was an enrichment of transcripts containing AP-2ε, HEYL, HES4, and TCFL5 transcription factor binding sites. It was found that the lncRNA content was similar between samples, with PCBP1-AS1, TEX41, and PVT1 being the top represented transcripts. There were 286 miRNAs found in the salEV samples. The pathways predicted to be affected by the top represented miRNAs include Hippo signaling, TGF-β signaling, Wnt signaling, FoxO signaling, ErbB signaling, axon guidance, and mTOR signaling. The inventors could detect retrotransposon transcripts from LINE, SINE, and LTR elements in salEVs. Retrotransposons are known to be derepressed with age and the onset of neurodegenerative diseases. When compared to blood-derived EVs, salEVs showed greater representation of transcripts associated with neurodegenerative pathways. The presence of these transcripts and retrotransposons in salEVs indicate that saliva may be used to screen for neurodegenerative diseases.

### BRIEF DESCRIPTION OF THE FIGURES

The present invention may be more readily understood by reference to the following figures, wherein:

FIGS. 1A-1C provide graphs and images showing the characterization of salEVs. A) mean size and concentration of salEVs determined by a Nanosight analysis. B) transmission electron micrographs of a representative salEV sample. Note the presence of a lipid bilayer encompassing the EVs. C) Western blot analysis of salEVs. CD9, CD81 and CD63 are tetraspanins are known EV markers, TSG101 and PDCD6IP are cytoplasmic proteins with membrane binding ability, HSP70 is a common EV cargo protein, APOA1 is associated with lipoprotein particles that commonly contaminate EV preparations.

FIGS. 2A-2D provide graphs showing the RNA-seq analysis of salEVs. A) the percentage of reads that uniquely or multi-map to the human genome. B) Transcript types found in salEVs. C) KEGG pathways associated with messenger RNA transcripts found in salEVs. Pathways in bold are known to be dysregulated in AD. D) transcription factor binding sites enriches in promoter regions of mRNA transcripts in salEVs.

FIGS. 3A-3C provide graphs and images showing the non-coding RNAs found in salEVs. A) the distribution and types of repetitive elements found in salEVs. B) The top 20 most abundant miRNAs found in salEVs. miRNAs in bold are differentially expressed in AD and inflammation. C) Pathways predicted to be targeted by miRNAs found in salEVs. Pathways in bold are known to be dysregulated in AD.

FIGS. 4A & 4B provide images showing comparison of salEVs to blood-derived EVs. A) comparison of the top represented transcripts in salEVs and bEVs. * denotes highly represented genes in salEVs that show lower abundance in bEVs. B) Representation of the top 1000 transcripts found in SalEVs and bEVs. The blue vertical bar to the left of each plot indicates transcripts associated with the Alzheimer's disease KEGG pathway hsa05010. Pathway transcripts are indicated with a dark blue line. Note the larger number of pathway-associated transcripts in salEVs.

FIG. 5 provides a graph showing the top 20 most abundant lncRNAs in each sample. The top 20 lncRNAs show a high degree of similarity across samples. The most abundant lncRNA, PCBP1-AS1 was the same across 14 of the 15 samples. The lncRNAs are color-coded to show similarity across the samples.

### DETAILED DESCRIPTION OF THE INVENTION

The present disclosure provides a method of identifying subjects having an increased risk of having or developing Alzheimer's disease (AD) or an Alzheimer's disease-related disorder (ADRD). The method includes the steps of obtaining a saliva sample from the subject; isolating extracellular vesicles from the saliva sample; determining the level of a protein or RNA biomarker present in the extracellular vesicles; comparing the level of the biomarker to a control level; and determining that the subject has an increased risk of having or developing AD or an ADRD if the biomarker level is altered relative to that of the control level. A method of evaluating treatment of AD or an ADRD in a subject receiving treatment for AD or an ADRD is also provided.

### Definitions

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

The term “biomarker” as used herein refers to an mRNA, microRNA, or protein used as an indicator of a biological state or condition, such as Alzheimer's disease (AD) or an Alzheimer's disease-related disorder (ADRD).

As used herein, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age. Thus, adult, juvenile, and newborn subjects are intended to be covered. As used herein, patient or subject may be used interchangeably and can refer to a subject afflicted with a disease or disorder (e.g. Alzheimer's disease). The term patient or subject includes human and veterinary subjects.

The term “polynucleotide” as used herein refers to a nucleic acid sequence including DNA and RNA, and can refer to markers which are either double stranded or single stranded. Polynucleotide can also refer to synthetic variants with alternative sugars like the LNA.

The term “expression” as used herein refers to the presence of biomarkers, such as the presence of a category of RNAs. A “change in expression” refers to a difference in the measurement of the biomarkers in a sample and known, controlled measurements of the biomarkers, such as the difference in level of a category of RNAs expressed and the normal or control level of that category of RNA.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “the microRNA” includes reference to one or more microRNA, and so forth.

### Methods of Identifying Subjects Having an Increased Risk of Having or Developing Alzheimer's Disease (AD) or an Alzheimer's Disease Related Disease

One aspect of the present invention provides a method of identifying subjects having an increased risk of having or developing Alzheimer's disease (AD) or an Alzheimer's disease-related disease (ADRD). The method includes obtaining a saliva sample from the subject; isolating extracellular vesicles from the saliva sample; determining the level of a protein or a category of RNA present in the extracellular vesicles; comparing the level of the protein or a category of RNA (i.e., biomarker) to a control level; and determining that the subject has an increased risk of having or developing AD or an ADRD if the biomarker level is altered relative to that of the control level. In some embodiments, the method is used to identify a subject having an increased risk of having or developing Alzheimer's disease.

In some embodiments, the protein or category of RNA (i.e., biomarker) is up-regulated in subjects having an increased risk of having or developing a neurodegenerative disease. In further embodiments, the biomarker is down-regulated in subjects having an increased risk of having or developing AD or an ADRD.

The degree of change (e.g., up-regulation or down-regulation) in the expression level of a biomarker when determined as indicating the presence of AD or an ADRD can be, for example, preferably 25% or more, 50% or more, 75% or more, or 100% or more as a percentage relative to a control, and the degree of a decrease in the expression level of the biomarker when determined as indicating the presence of AD or an ADRD can be, for example, preferably 25% or more, more preferably 50% or more, still more preferably 75% or more as a percentage relative to a control.

### Categories of RNA

The biomarker can be a protein or a category of RNA. In some embodiments, the method comprises determining the level of a protein. Proteins, as is known by those skilled in the art, are large biomolecules comprising one or more long chains of amino acid residues, such as enzymes, receptors, immune components such as antibodies, hormones, or structural components of body tissues.

In further embodiments, the method comprises determining the level of a category of RNA. Categories of RNA are groups (i.e., a plurality) of RNA that have a common feature, such as overall structure, a particular function, or shared nucleotide motifs such as transcription factor binding motifs. Examples of categories of RNA include mRNA, microRNA, lncRNA, and retrotransposon RNA. Other categories of RNA include RNA associated with certain biochemical pathways, such as signaling or pathways known to be associated with neurodegenerative diseases such as Alzheimer's disease.

In some embodiments, the category of RNA is mRNA. mRNA, also known as messenger RNA and protein-coding RNA, is a single-stranded molecule of RNA that corresponds to the genetic sequence of a gene, and is read by a ribosome in the process of synthesizing a protein. mRNA can be modified by 5′ cap addition and polyadenylation, and can include both coding and untranslated regions. mRNA molecules vary depending on the gene they transcribe, but typically range from about 300 to about 3,000 nucleotides. In some embodiments, the mRNA are associated with pathways involved in neurodegenerative conditions. In further embodiments, the mRNA are enriched for transcription factor binding sites, such as AP-2ε, HEYL, HES4, and TCFL5.

In some embodiments, the category of RNA is microRNA. The term “microRNAs” or “miRNAs” as used herein refers to a class of small RNAs typically between 15 and 30 nucleotides long. microRNAs can refer to a class of small RNAs that play a role in gene regulation. In a preferred embodiment, a microRNA refers to a human, small RNA of 20, 21, 22, 23, 24, 25, or 26 nucleotides long. In some embodiments, the microRNA as associated with various biochemical pathways such as Hippo signaling, TGF-0 signaling, Wnt signaling, FoxO signaling, ErbB signaling, axon guidance, and mTOR signaling.

In some embodiments, the microRNA is selected from the group consisting of hsa-miR-498-3p, hsa-miR-614, hsa-miR-107, hsa-miR1249-5p, and hsa-miR-519-3p. These sequences are shown in Table 1.

TABLE 1
microRNA sequences

| Identifier | Nucleotide sequence |
|---|---|
| hsa-miR-498-3p | AAAGCACCUCCAGAGCUUGAAGC |
| | (SEQ ID NO: 1) |
| hsa-miR-614 | GAACGCCUGUUCUUGCCAGGUGG |
| | (SEQ ID NO: 2) |
| hsa-miR-107 | AGCAGCAUUGUACAGGGCUAUCA |
| | (SEQ ID NO: 3) |
| hsa-miR1249-5p | AGGAGGGAGGAGAUGGGCCAAGUU |
| | (SEQ ID NO: 4) |
| hsa-miR-519-3p | CAAAGUGCCUCCCUUUAGAGUG |
| | (SEQ ID NO: 5) |

In some embodiments, the category of RNA is lncRNA. A lncRNA, or long non-coding RNA, is a type of RNA molecule that is more than 200 nucleotides long and does not code for proteins. These molecules act as key regulators in cellular processes, including development, differentiation, and the response to diseases like cancer and neurodegenerative disorders. LncRNAs function in diverse ways, such as by interacting with DNA and proteins to regulate gene expression, with roles in both the nucleus and cytoplasm. In some embodiments, the lncRNA is PCBP1-AS1, TEX41, and/or PVT1.

In some embodiments, the category of RNA is retrotransposon RNA. Retrotransposons are mobile elements which move in the host genome by converting their transcribed RNA into DNA through reverse transcription. There are two main types of retrotransposons, long terminal repeats (LTRs) and non-long terminal repeats (non-LTRs). LTR retrotransposons are characterized by their long terminal repeats (LTRs), which are present at both the 5′ and 3′ ends of their sequences. These LTRs contain the promoters for these transposable elements (TEs), are essential for TE integration, and can vary in length from just over 100 base pairs (bp) to more than 1,000 bp. Non-LTR retrotransposons contain genes for reverse transcriptase, RNA-binding protein, nuclease, and sometimes ribonuclease H domain but they lack the long terminal repeats, and cannot carry out reverse transcription. The two main types of non-LTR retrotransposons are LINE (long interspersed nuclear elements) and SINE (short interspersed nuclear elements). See Richardson et al., Microbiol Spectr., 3(2):MDNA3-0061-2014 (29015). In some embodiments, the category of RNA comprises LINE and SINE retrotransposons.

### Saliva Samples and Extracellular Vesicles

The method includes the step of analyzing a saliva sample from a subject. Saliva and/or liquid from the oral cavity of a subject contains, among other things, water, proteins, electrolytes, mucus, serum and serum components, blood cells, mucous membrane cells as well as microorganisms and antibodies. A saliva sample can be obtained by collected saliva in a receiving device such as a cup (e.g., by having the subject spit into a cup) or using any other receiving device such as a cotton swab which can be used to obtain saliva from within the oral cavity. The term “receiving device” refers to a construction which is configured particularly to remove saliva from the subject (e.g., a human or animal), by preferably oral contact with a subject, particularly by chewing and/or biting of the receiving device, and/or for sucking up and/or absorbing saliva and/or sample material from the subject and/or for preparing a saliva sample.

A saliva sample may be fresh or one that has been previously obtained and stored. Samples can be stored for varying amounts of time, such as being stored for an hour, a day, a week, a month, or more than a month. The saliva sample may be expressly obtained for the assays of this invention or obtained for another purpose which can be sub-sampled for the assays of this invention.

The method includes the step of isolating extracellular vesicles from the saliva sample. Extracellular vesicles (EVs) are lipid bound vesicles secreted by cells into the extracellular space. The three main subtypes of EVs are microvesicles (MVs), exosomes, and apoptotic bodies, which are differentiated based upon their biogenesis, release pathways, size, content, and function. The content of EVs consists of lipids, nucleic acids, and proteins, and in particular proteins associated with the plasma membrane, cytosol, and those involved in lipid metabolism. Yanez-Mo et al., J Extracell Vesicles, 4:27066 (2015).

A variety of methods are known to those skilled in the art for isolating extracellular vesicles. In some embodiments, exosomes can be isolated by ultracentrifugation-based methods. Tauro et al., Methods, 56:293-304 (2012). Additional methods have been developed based on isolation by size, immunoaffinity capture, and precipitation of exosomes, including density gradient isolation, precipitation kits, Exosome Isolation kits (e.g., ExoMir® Kit, Biotium™) Immunoprecipitation, Multiplexed ExoSearch Chip, and Acoustic Nanofilter. Doyle L. and Wang M., Cells, 8(7): 727 (2019).

### Diagnosing Alzheimer's Disease or an Alzheimer's Disease-Related Disorder

One aspect of the invention provides a method of diagnosing Alzheimer's disease (AD). Alzheimer's disease (AD) is a chronic neurodegenerative disease that results in the loss of neurons and synapses in the cerebral cortex and certain subcortical structures, resulting in gross atrophy of the temporal lobe, parietal lobe, and parts of the frontal cortex and cingulate gyrus. Wenk G., The Journal of Clinical Psychiatry. 64 Suppl 9: 7-10 (2003). Alzheimer's disease is usually diagnosed based on the person's medical history, history from relatives, and behavioral observations. The presence of characteristic neurological and neuropsychological features and the absence of alternative conditions is supportive. Advanced medical imaging with computed tomography (CT) or magnetic resonance imaging (MRI), and with single-photon emission computed tomography (SPECT) or positron emission tomography (PET) can be used to help exclude other cerebral pathology or subtypes of dementia.

In some embodiments, the method is used to diagnose an Alzheimer's disease-related disorder (ADRD), also known as Alzheimer's related dementia. See Corriveau et al., Neurology, 89(23):2381-2391 (2017). Alzheimer's disease-related disorders include dementia-causing neurodegenerative disorders such as frontotemporal dementia (FTD), Parkinson's disease (PD), dementia with Lewy bodies (DLB), and amyotrophic lateral sclerosis (ALS), which are a phenotypically diverse set of disorders that share some degree of overlap in terms of risk factors and common cellular pathways, such as those involved in neuroinflammation. ADRDs share many cognitive symptoms and brain changes with AD and each other that can make them difficult to distinguish and differentially diagnose. Alzheimer's disease related disorders typically involve proteinopathies involving β-amyloid protein and tau protein. Other examples of Alzheimer's disease-related disorders include Huntington disease and Prion disease.

In some embodiments, the method of diagnosis further comprises treating the subject identified as having an increased risk of having or developing AD or ARDR. A number of methods of treating Alzheimer's disease or an Alzheimer's disease-related disorder are described herein. Examples of drugs used to treat Alzheimer's disease and Alzheimer's disease-related disorders include antihypertensives, statins, antidepressants, antidiabetic agents, proton pump inhibitors, benzodiazepines, and anti-inflammatory agents. Treatment of Alzheimer's disease can also include non-drug therapies such as changes in diet and exercise, and mental exercises.

### Methods of Evaluating Treatment of Alzheimer's Disease (AD) or an Alzheimer's Disease-Related Disorder (ADRD)

A further aspect of the present invention provides a method of evaluating treatment of Alzheimer's disease (AD) or an Alzheimer's disease-related disorder (ADRD) in a subject receiving treatment for AD or ADRD. The method includes obtaining a saliva sample from the subject; isolating extracellular vesicles from the saliva sample; determining the level of a biomarker (i.e., a protein or category of RNA) present in the extracellular vesicles; comparing the level of the biomarker to a control level corresponding to the biomarker level before treatment, and determining if the treatment is effective if AD or ADRD if the biomarker level is altered relative to that of the control level. In some embodiments, wherein the method is used to identify a subject being treated for Alzheimer's disease.

A number of methods for treating AD and ADRD have been identified. See Bednar, M., Prog Mol Biol Transl Sci., 168:289-296 (2019), and Thunell et al., Alzheimers Dement., 17(1):41-48 (2021), the disclosures of which are incorporated herein by reference. Cholinesterase inhibitors such as galantamine, rivastigmine, and donepezil can be used to treat symptoms of Alzheimer's disease, while immunotherapy using lecanemab and aducanumab can be used to treat AD by targeting β-amyloid. The NMDA antagonist memantine and the antipsychotic brexpiprazole can also be used to treat Alzheimer's disease. Methods for treating ADRD vary depending on the specific disease. For example, levodopa (alone or in combination with carbidopa) can be used to treat Parkinson's disease, while riluzole, edaravone, and sodium phenylbutyrate-taurursodiol can be used to treat ALS. A number of agents have been shown to be useful for treating Lewy Body Dementia. See Skylar-Scott, I and Sha, S., T, Curr Neurol Neurosci Rep., 23(10):581-592 (2023). The small molecule CT1812 has shown the ability to treat multiple types of dementia by displacing toxic protein aggregates at synapses. See Kaur et al., Clin Ther., 46(11):e21-e28 (2024).

In some embodiments, the protein or category of RNA (i.e., biomarker) is up-regulated in subjects where treatment of AD or ADRD is effective, while in other embodiments the biomarker is down-regulated in subjects where treatment of AD or ADRD is effective.

The biomarker can be a protein or a category of RNA. In some embodiments, the method comprises determining the level of a protein. In further embodiments, the method comprises determining the level of a category of RNA, such as an mRNA, a microRNA, a lncRNA, or a retrotransposon RNA. In yet further embodiments, the method comprises determining the level of a microRNA. In some embodiments, the microRNA is selected from the group consisting of hsa-miR-498-3p, hsa-miR-614, hsa-miR-107, hsa-miR1249-5p, and hsa-miR-519-3p.

In some embodiments, the RNA is detected using a polynucleotide probe. The term “probe” as used herein refers to a polynucleotide sequence that will hybridize to a complementary target sequence. In one example, the probe hybridizes to a microRNA sequence. The probes provided herein have nucleotide sequences that have 90% sequence identity to polynucleotide sequences that are the complement of a microRNA or mRNA having altered expression as a result of Alzheimer's disease (AD) or an Alzheimer's disease-related disorder (ADRD). A method of detecting AD or an ADRD can use at least one, at least two, at least three, at least four, at least five, at least six, at least seven, at least eight, at least nine, at least ten, at least eleven, at least twelve, or at least thirteen probes.

A person skilled in the art will appreciate that a number of methods can be used to detect or quantify the level of a category of RNA within a sample, including microarrays, PCR (including quantitative RT-PCR), nuclease protection assays, in situ hybridization, nanoString analysis, and microfluidics devices.

The detection method is not particularly limited provided that it can measure the level of the microRNA or mRNA whose expression changes in response to AD or an ADRD. In the example of a microarray, the method involves labeling the RNA extracted from a tissue with a label (preferably a fluorescent label), contacting the RNA with a microarray to which a probe consisting of a polynucleotide (preferably DNA) consisting of a nucleic acid sequence complementary to the microRNA or mRNA to be identified or a part thereof is fixed for hybridization, washing the microarray, and measuring the expression level of the remaining microRNAs or mRNA on the microarray.

The type of the nucleotide of the nucleic acid sequence is not particularly limited provided that it can specifically hybridize to the microRNA or mRNA biomarker The length of the part of the polynucleotide is not particularly limited provided that it specifically hybridizes to the predetermined microRNA or mRNA according to the present invention; however, it is preferably 10 to 100 mers, more preferably 10 to 40 mers in view of securing the stability of hybridization. The polynucleotide or a part thereof can be obtained by chemical synthesis or the like using a method well known in the art.

The array to which the polynucleotide or a part thereof is fixed is not particularly limited; however, preferred examples thereof can include a glass substrate and a silicon substrate, and the glass substrate can be preferably exemplified. A method for fixing the polynucleotide or a part thereof to the array is not particularly limited; a well-known method may be used.

The quantitative PCR method is another method suitable for detecting polynucleotide biomarkers, and is not particularly limited provided that it is a method using a primer set capable of amplifying the sequence of the microRNA or mRNA and can measure the expression level of the present microRNA or mRNA; conventional quantitative PCR methods such as an agarose electrophoresis method, an SYBR green method, and a fluorescent probe method may be used. However, the fluorescent probe method is most preferable in terms of the accuracy and reliability of quantitative determination. Quantitative PCR also requires that an adaptor be added to the 3′ end of the RNA.

The primer set for the quantitative PCR method means a combination of primers (polynucleotides) capable of amplifying the sequence of the RNA (e.g., microRNA or mRNA). The primers are not particularly limited provided that they can amplify the sequence of the microRNA or mRNA; examples thereof can include a primer set consisting of a primer consisting of the sequence of a 5′ portion of the sequence of an RNA nucleotide (forward primer) and a primer consisting of a sequence complementary to the sequence of a 3′ portion of the microRNA (reverse primer). Here, the 5′ means 5′ to the sequence corresponding to the reverse primer when both primers were positionally compared in the sequence of a mature microRNA; the 3′ means 3′ to the sequence corresponding to the forward primer when both primers were positionally compared in the sequence of an RNA nucleotide. Primers are typically 18-24 nucleotides in length.

In some embodiments, a fluorescent probe is used. The fluorescent probe is not particularly limited provided that it comprises a polynucleotide consisting of a nucleic acid sequence complementary to the sequence of the RNA or a part thereof; preferred examples thereof can include a fluorescent probe capable of being used for the TaqMan™ probe method or the cycling probe method; the fluorescent probe capable of being used for the TaqMan™ probe method can be particularly preferably exemplified. Examples of the fluorescent probe capable of being used for the TaqMan™ probe method or the cycling probe method can include a fluorescent probe in which a fluorochrome is labeled 5′ thereof and a quencher is labeled on 3′ thereof. The fluorochrome, quencher, donor dye, acceptor dye used or the like used with a fluorescent probe are commercially available.

Conventional techniques of molecular biology, microbiology and recombinant DNA techniques, are within the skill of the art. Such techniques are explained fully in the literature. See, e.g., Sambrook, Fritsch & Maniatis, 2012, Molecular Cloning: A Laboratory Manual, Fourth Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

A person skilled in the art will appreciate that a number of detection agents can be used to determine the expression of the biomarkers. For example, to detect the RNA of a particular category of RNA, probes, primers, complementary polynucleotide sequences or polynucleotide sequences that hybridize to the RNA products can be used. In some embodiments, reverse complementary polynucleotides serve as probes for microRNA markers.

The levels of protein biomarkers may be determined by any of a variety of standard protein analytic methods known in the art. These methods include absorbance, protein assays (e.g., DC protein assay, non-interfering protein assay, BCA protein assay, and the Lowry assay), gel electrophoresis (e.g., SDS-PAGE gel purification), a protein immunoblot (e.g., western blot), chromatography (e.g., size exclusion chromatography, ion exchange chromatography, and affinity chromatography), precipitation, ultracentrifugation, an immunoassay, such as an enzyme-linked immunosorbent assays (ELISA), mass spectrometry, and other common techniques known to one of ordinary skill in the art. In some embodiments, the assay used allows one to simultaneously determine the levels of multiple biomarkers in a saliva sample. For example, an immunoassay using fluorescent imaging may be used, where the antibodies used to detect multiple protein biomarkers that emit at different fluorescent wavelengths so that their fluorescent signals can be easily distinguished.

An immunoassay is an assay that uses a binding ligand (e.g., an antibody) to specifically bind an antigen (e.g., a biomarker). An immunoassay is characterized by the use of specific binding properties of a particular binding ligand to isolate, target, and/or quantify the antigen. While use of aptamers as the binding ligand is not the use of a component of the immune system, assays using aptamers are nonetheless referred to herein as “immunoassays.” Specific binding to a binding ligand (e.g., antibody) under such conditions may require an antibody that is selected for its specificity for a particular protein. For example, polyclonal or monoclonal antibodies raised to a biomarker from specific species such as rat, mouse, or human can be selected that are specifically reactive with that biomarker and not with other proteins, except for polymorphic variants and alleles of the biomarker. This selection may be achieved by subtracting out antibodies that cross-react with the biomarker molecules from other species. Immunoassays can be run using a variety of different formats, including competitive homogenous immunoassays, heterogeneous immunoassays, one-site non-competitive immunoassays, and two-site non-competitive immunoassays.

In some embodiments, the step of determining the level of a protein or category of RNA present in the extracellular vesicles is carried out using a point-of-care device. The point-of-care device includes a microfluidic point-of-care platform, and a mobile device configured to access a communications network and having a processor configured to access a camera configured to acquire fluorescent images is used to determine the levels of one or more biomarkers. Integration of a mobile device into the detection system enables immediate processing of the results without requiring the use of a conventional, bulky and expensive spectrophotometer. Methods for integrating a mobile device to a microfluidic assay system are known in the art. See U.S. Patent Publication US 2014/0242612, the disclosure of which is incorporated herein by reference. The mobile device can include an application stored on and executed by a processor of the mobile device to analyze the signals detected from the labeled binding ligands in the microfluidic device.

Use of a microfluidic point-of-care platform allows for rapid detection of biomarkers using a readily portable, and in some cases disposable, device. A wide variety of point-of-care microfluidic devices are commercially available (Han KN1, Li C A, Seong G H, Annu Rev Anal Chem (Palo Alto Calif), 6:119-41 (2013)), and are particularly useful for diagnosing disease in low resource settings. Sharma et al., Biosensors (Basel). 5(3):577-601 (2015).

An example has been included to more clearly describe a particular embodiment of the invention and its associated cost and operational advantages. However, there are a wide variety of other embodiments within the scope of the present invention, which should not be limited to the particular examples provided herein.

### EXAMPLE

Use of mRNA and miRNA of Salivary Extracellular Vesicles to Identify Individuals with Neurodegenerative Diseases

In this study we determined that RNA transcripts associated with neurodegenerative pathways can be detected in salEVs in people over the age of 65. We also identified non-protein coding sequences that are associated with neurodegenerative conditions. Detecting changes in salEV RNA content may allow for the discovery of biomarkers of neurodegeneration early in the disease process, allowing for treatments to begin at an earlier point when therapeutics may be more effective. This study sets the stage for detection of biomarkers of neurodegeneration in salEVs.

### Materials and Methods

### Participants

Whole saliva specimens were collected from healthy older (65+) adult participants (N=15). enrolled in a longitudinal study of neurodegeneration and cognitive aging at the Rhode Island Hospital Alzheimer's Disease and Memory Disorders Center in Providence, RI USA (Table 1). Healthy volunteers were excluded from the study if they had signs of dementia, major primary psychiatric illnesses, congenital brain disorders, intellectual disability, and history of traumatic brain injury. Informed consent was obtained from participants before enrollment in the study. The Brown University Health institutional review board approved the study in accordance with good clinical practice guidelines and the Declaration of Helsinki.

TABLE 1
Participant Characteristics

| | N = 15 | |
|---|---|---|
| Age, (mean), years, (SD) | 72 | (3.4) |
| Sex, % Female | 80 | |
| Race and Ethnicity, (n) | 15 White, | |
| | non-Hispanic | |
| Montreal Cognitive Assessment, | 27 | (1.7) |
| (mean), total score | | |
| Mini-mental State Exam, | 27.9 | (1.9) |
| (mean), total score | | |
| APOE proteotype, (n) | | |

| | |
|---|---|
| E2/E3 | 3 |
| E3/E4 | 4 |
| E3/E3 | 8 |

### Saliva Collection

Participants were instructed to abstain from eating, chewing gum or smoking at least 2 hours before the saliva collection. After signing consent, a research assistant described the procedure and provided a sterile polypropylene collection and storage kit (SputEm™, Simport). Participants were encouraged to produce a minimum of 5 ml saliva. Within 2 minutes of completion, the collection tube was sealed and stored at −80° C. until analyzed.

### EV Isolation

The protocol for salEV isolation was adapted and modified from the exosome isolation protocols of Michael et al., Oral Dis., 16(1):34-8 (2010) and Lässer et al. J Transl Med., 9:9 (2011). Briefly, samples were kept frozen at −80° C. until processed. Frozen saliva was thawed in 37° C. water bath with DNase I (Roche) at 100 μg per milliliter of saliva for 20 minutes. Collected saliva (5 ml) was diluted with 1× phosphate buffered saline (PBS, Gibco) to a final volume of 25 ml. The saliva suspension was centrifuged at 2000×g for 10 minutes at 4° C. to remove large debris. The supernatant was transferred to another tube and centrifuged at 17,000×g for 15 minutes at 4° C. to further remove unwanted organelles and cell fragments. Following the initial centrifugation steps, the supernatant was transferred to sterile 36 ml PA tubes (Sorvall) for ultracentrifugation at 120,000×g for 70 minutes at 4° C. in a Surespin630 rotor (Sorvall) with full brake and acceleration (both set at 9). Following ultracentrifugation, the aqueous layer, which is viscous in whole saliva samples, was removed and the pellet containing the EVs was washed with phosphate buffered saline (PBS, Thermo Fisher Scientific) and ultracentrifuged again at 120,000×g for 70 minutes at 4° C. For long term storage at −80° C., EV pellet resuspended in 1% Dimethyl sulfoxide (DMSO, Sigma) in PBS.

Measurement of Particle Size and Concentration Distribution with NanoSight

Nanoparticles in the salEV suspensions were analyzed using the NanoSight NS500 instrument (NanoSight Ltd). The analysis settings were optimized and kept constant between samples, and each video was analyzed to give the mean, mode, median and estimated concentration for each particle size. Samples were measured at 1:20 dilution, yielding particle concentrations in the range of 1×10⁸ particles ml⁻¹ in accordance with the manufacturer's recommendations. All samples were analyzed in triplicate.

### RNA Isolation

EV pellets after ultracentrifugation were lysed using Trizol (Invitrogen). RNA was isolated using Trizol according to the manufacturer's protocol and dissolved in Nuclease-Free water (Ambion). Briefly, samples were centrifuged following the addition of chloroform and the aqueous phase containing RNA was collected. RNA was precipitated using isopropanol and collected by centrifugation at 12,000×g for 15 min. The RNA pellet was washed with 70% ethanol and resuspended in RNase free water. Quantification was done using a Nanodrop 1000.

### Protein Isolation

SalEVs were lysed on ice for 30 minutes using 10λ cell lysis buffer (Cell Signaling) supplemented with Halt Protease and Phosphatase Inhibitor Cocktail (100×) (Thermo Fisher Scientific) following the manufacturer's recommendations. The lysates were then centrifuged at 14,000×g for 15 minutes at 4° C., and the supernatant was collected. Protein concentration of EV lysates was quantified using the Pierce™ BCA Protein Assay Kit (Thermo Fisher Scientific).

### Western Blots

SalEV lysates were loaded onto 4-20% Mini-PROTEAN® TGX™ Precast Protein Gels (Bio-Rad) and separated by SDS-PAGE. The proteins were then transferred to PVDF membranes (Bio-Rad) using the Trans-Blot Turbo Transfer System (Bio-Rad). The membranes were incubated overnight at 4° C. with the following primary antibodies: CD81 (System Biosciences, #EXOAB-CD81A-1), CD9 (System Biosciences, #EXOAB-CD9A-1), CD63 (System Biosciences, #EXOAB-CD63A-1), TSG101 (Thermo Fisher Scientific, #MA1-23296), PDCD6IP (Thermo Fisher Scientific, #50-167-7058), GAPDH (Thermo Fisher Scientific, #PA1-987), heat shock protein 70 (HSP70; Santa Cruz, #sc-59560), and APOA1 (Santa Cruz, #sc-376818). Signal on PVDF membranes were developed with SuperSignal™ West Atto Ultimate Sensitivity Substrate (Thermo Fisher Scientific) and captured using the ChemiDoc™ MP Imaging System (Bio-Rad).

### TEM

Transmission electron microscopy (TEM) was performed on isolated salEVs fixed with 2% paraformaldehyde, placed on 200 mesh copper formvar carbon coated grids (Electron Microscopy Science) and left to adhere for 20 min. EVs were negatively stained with UAR-EMS (Electron Microscope Science). Grids were viewed on a Philips 410 Transmission Electron Microscope equipped with an Advantage HR CCD camera. Images were acquired with Advanced Microscopy Techniques imaging software.

### RNA Sequencing

A total of 5 μl of RNA (regardless of RNA concentration) isolated from salEVs was sent to Azenta for low-input RNA sequencing. The fastq files were groomed using fastp (Chen S., Imeta, 2(2):e107 (2023)) to remove adaptors, filter poor quality reads, and remove duplicates. The resulting files containing high quality reads were mapped to the human genome HG38 using STAR. Dobin et al., Bioinformatics, 29(1):15-21 (2013). The identity of the features represented by the mapped reads were determined by FeatureCounts (Liao et al., Bioinformatics, 30(7):923-30 (2014)) utilizing the Gencode Release 43 (GRCh38.p13) transcript annotation file. The pathways associated with the identified coding transcripts were determined using the Kyoto Encyclopedia of Genes and Genomes Database for Annotation, Visualization, and Integrated Discovery database. The RNA was classified by type (i.e. protein coding, lncRNA, etc. . . . ) and transcription factor binding sites were identified using ShinyGO 0.77. Ge et al., Bioinformatics, 36(8):2628-9 (2020). We used 117 publicly available blood EV RNA-seq datasets from healthy control donors that were part of GSE133684. Yu et al., Gut, 69(3):540-50 (2020). The healthy control participants ranged from 41-91 years in the blood datasets.

miRNA Analysis

The miRNA content of salEVs was determined using the Nanostring platform. Between 12.87-100 ng/sample were run on a NanoString Human v3 miRNA Assay at Boston Children's Hospital IDDRC Molecular Genetics Core. Representation levels of 827 miRNAs were determined using the nCounter software (Nanostring). The mirPath program on the Diana Tools website was used to predict the physiological pathways that would be affected by alterations in the miRNA cytoplasmic concentrations. Vachos et al., Nucleic Acids Res., 43(W1):W460-6 (2015).

### Repetitive Element Analysis

Expression levels of repetitive elements in salEVs were determined using the TE-seq methods outlined in Kelsey et al., bioRxiv (2025). Briefly, the sequencing files described above for RNA sequencing were groomed with fastp as described above, then aligned to the telomere-to-telomere (T2T) human genome using STAR. The alignment files were processed with Telescope to determine the repetitive element counts in each sequencing dataset. Bendall et al., PLoS Comput Biol., 15(9):e1006453 (2019).

### Results

Characterization of EVs Isolated from Saliva

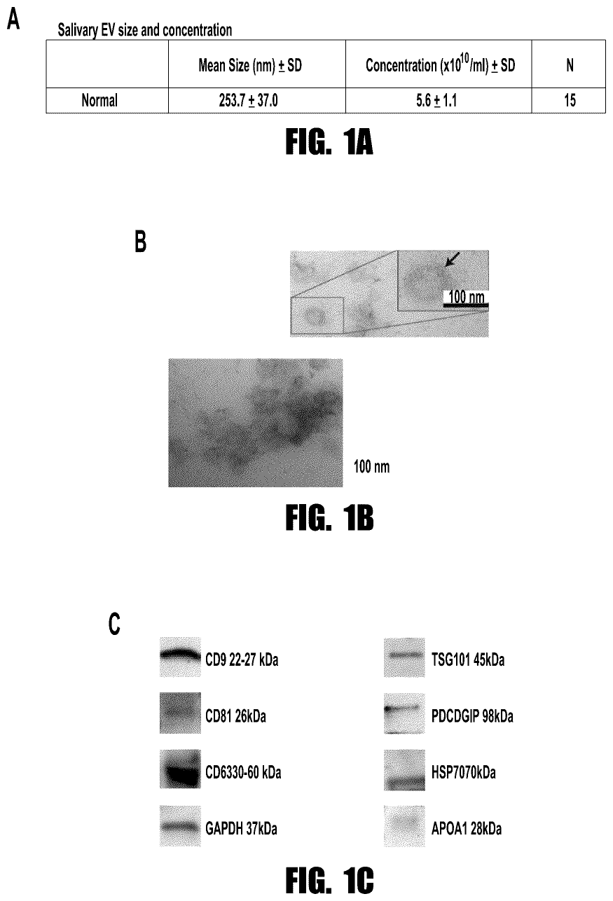

We collected whole saliva from 15 cognitively normal older adults (mean (SD), 72 (3.4) years) and isolated salEVs. Participants were non-Hispanic White and the majority (80%, n=12) were female (Table 1). To verify that the isolated particles are EVs, we characterized the vesicles in compliance with the minimal information for studies of extracellular vesicles 2023 (MISEV2023). Welsh et al., J Extracell Vesicles, 13(2):e12404 (2024). We performed Nanosight analysis on the salEV preparation (FIG. 1A). The particles in the salEV preparations were present at an average concentration of 5.6±1.1×10¹⁰/ml. Analysis of the size distribution of the salEV particles showed a mean diameter of 253.7±37.0 nm. The average particle size was >100 nm, indicating the preparation likely contained a combination of microvesicles (100 nm-1 μm) and exosomes (40-100 nm).

To further evaluate that the particles in the preparation are EVs, we used transmission electron microscopy (EM) to visualize the particles. The electron micrographs revealed cup-shaped membrane bound vesicles corresponding to both the microvesicle size range and the exosome size range, indicating the preparation contained a mixture of both vesicle types (FIG. 1B). Closer examination of the vesicles revealed that they were enclosed by a double membrane (FIG. 1B insert), which is consistent with both microvesicle and exosome membrane structure.

To determine that the membrane-bound vesicles seen by EM are EVs, we confirmed the presence of EV-specific markers. We performed Western blot analysis for the transmembrane tetraspanin markers CD9, CD63, and CD81, and found that all 3 tetraspanins were present in the preparations (FIG. 1C). We also verified the presence of cytosolic proteins with lipid or membrane protein binding ability by confirming the presence of tumor susceptibility gene 101 (TSG101) and programmed cell death 6-interacting protein (PDCD6IP) (FIG. 1C), both of which play a role in the endosomal sorting complexes required for transport (ESCRT) pathway that produces extracellular vesicles. Heat shock protein 70 (HSP70) is commonly found in EV cargo, and we verified the presence of HSP70 in the vesicle preparation (FIG. 1C). As a negative control, we examined levels of APOA1, which was minimally present indicating that the EV preparation was not significantly contaminated with lipoprotein particles (FIG. 1C). From these data we conclude that our EV preparations from saliva contained both microvesicles and exosomes.

### Transcriptomic Data

We isolated total RNA from the salEV preparations. The overall quality of the RNA was acceptable, considering that EV RNA quality is often considered poor due to the presence of fragmented and degraded RNAs. The RNA had an average 260/280 of 1.7±0.2 and an average total RNA concentration of 20.2±9.0 ng/μl and a small RNA concentration of 5.5 f 7.7 ng/μl.

mRNA Characterization

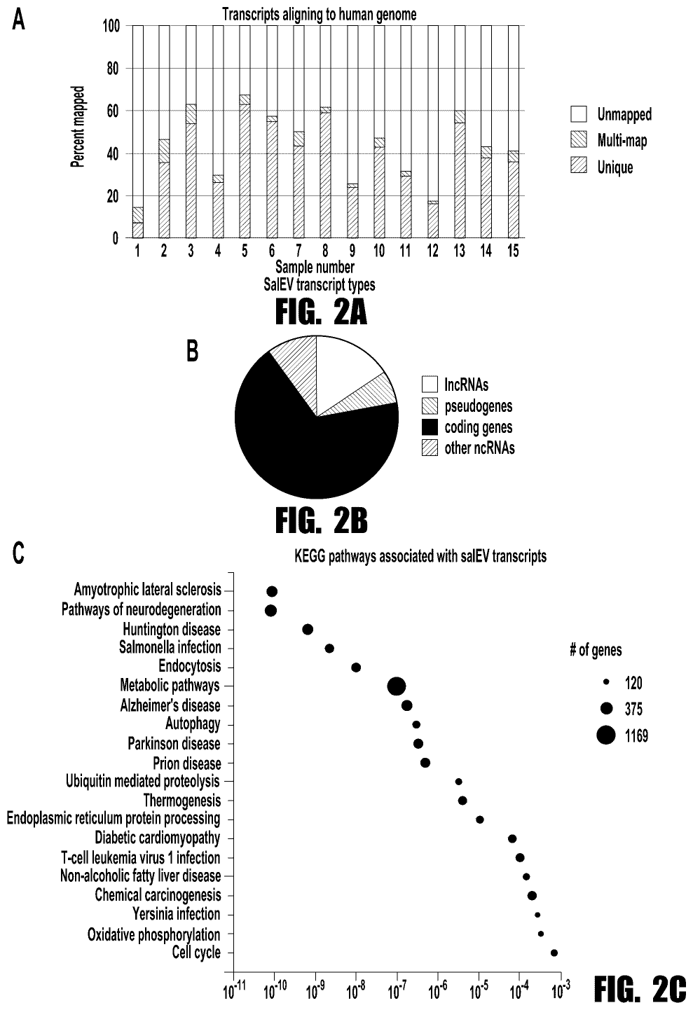

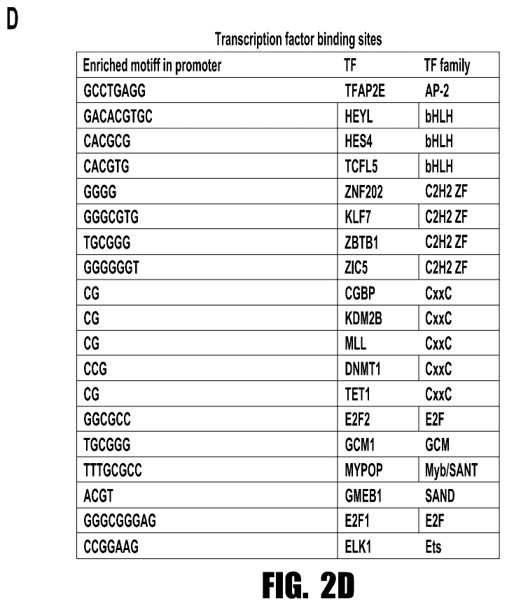

To characterize the larger transcripts present (>200 nt) we performed bulk-RNA-sequencing on salEVs from 15 samples. Since saliva is not sterile, and prokaryotic species are known to produce EVs (Mobarak et al., Cell Commun Signal, 22(1):80 (2024)), we examined the number of transcripts mapping to the human genome and the number mapping to bacterial genomes to determine the approximate representation of EVs originating from human cells in saliva. We found an average of 44.1% of the reads uniquely mapped and 4.3% of the reads multi-mapped to the human genome (FIG. 2A). The majority of the remaining reads multi-mapped to bacterial genomes. These results indicate that a significant portion of salEVs originate from human cells, and there is high enough representation to identify potential biomarkers for disease from salEVs.

Further analysis of the RNA content revealed that for most samples the majority of the transcripts >200 nucleotides originated from protein coding sequences. We examined the transcripts aligning to Ensembl annotations (vertebrate, release 96, (Aken, et al. Nucleic Acids Res., 45(D1):D635-D42 (2017)) and found an average of 19,541 distinct transcripts represented in each salEV preparation (FIG. 2B). On average, there were 12,213 protein coding gene transcripts represented in the data, or 68.1%. The next largest category of RNA was long, non-coding RNAs (lncRNAs), which represented approximately 3,457 transcripts in the preparations, or 15.7% of the transcripts. This is interesting since lncRNAs are known to play regulatory roles and the transfer of these transcripts to other cell types could affect cellular processes at distant locations. The remaining transcripts arose from pseudogenes and other non-coding RNAs. We were not able to quantify small RNAs such as miRNAs in these datasets, since the sequencing library preparation included size selection for transcripts >200 nt. Overall, we found a diverse array of RNA species present in salEVs, indicating that salEVs may affect a broad range of physiological processes.

We determined the top KEGG pathways represented by the protein coding transcripts. The top represented pathways were associated with neurodegeneration (FIG. 2C). These included Amyotrophic lateral sclerosis, Pathways of neurodegeneration—multiple diseases, Huntington disease, Alzheimer disease, Parkinson disease, and Prion disease. Our results suggest that transcripts associated with neurogenerative conditions are present in salEVs, and that salEVs could be used to screen individuals for risk of developing one of these conditions.

We analyzed the promotor regions of the genes identified in our sequencing results to determine if specific transcription factor (TF) target genes are overrepresented in our data. We found the most significantly represented TF enriched motifs in the promoters of coding transcripts were AP-2ε, which is a member of the AP-2 TF family, and HEYL, HES4, and TCFL5, which are members of the bHLH TF family (FIG. 2D). The bHLH TFs have been shown to act as transcriptional repressors and are abundantly expressed during development Heisig et al., PLoS genetics, 8(5):e1002728 (2012). These results suggest that the salEV cells of origin have increased levels of AP-2ε, and reduced levels of the bHLH TFs.

### Other RNA Types

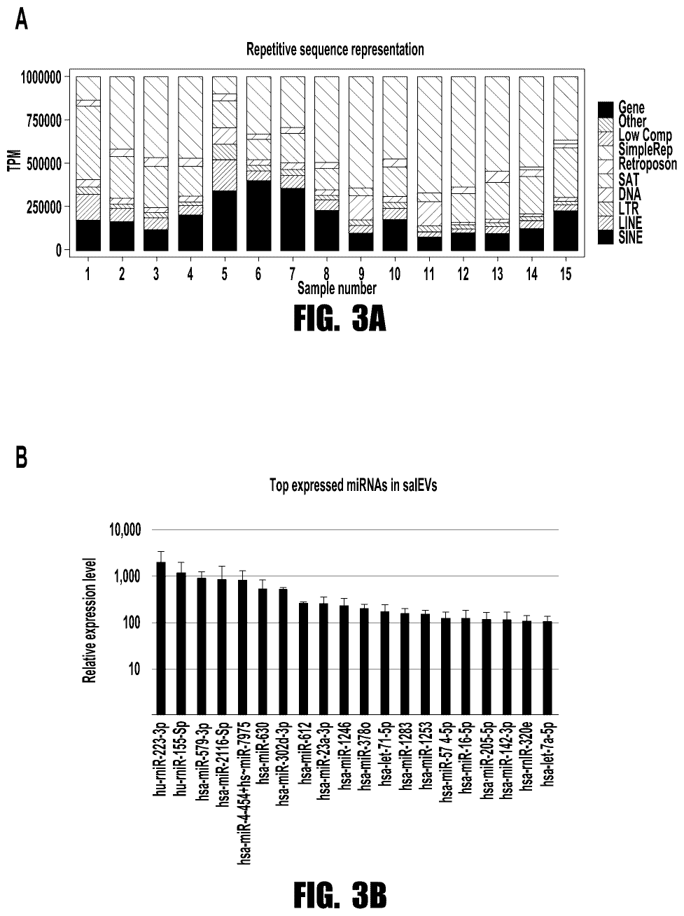

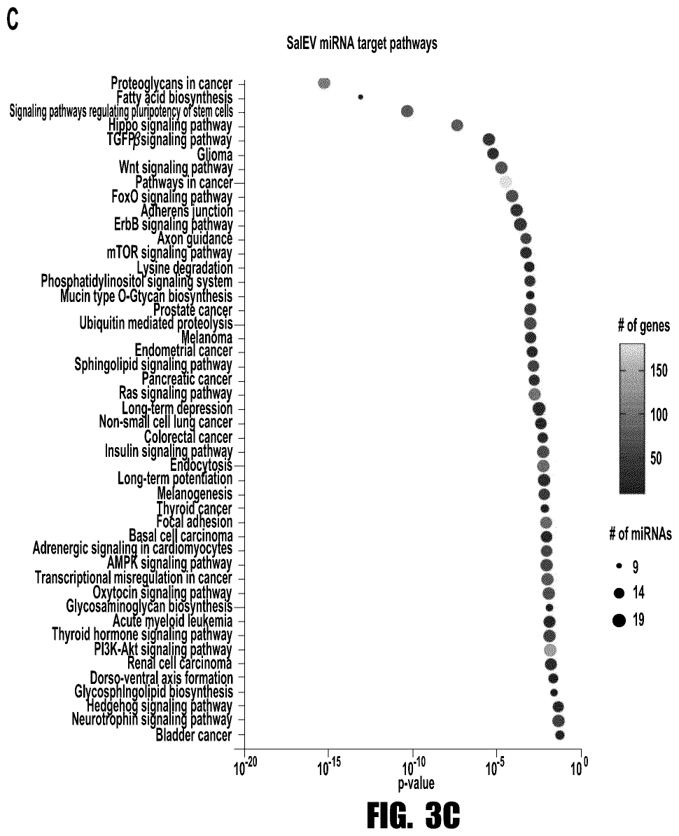

Repetitive elements represent a large portion of the human genome. While many repetitive elements are structural (centromeric and telomeric repeats), others have evolved from viruses and are capable of transposition within the genome. A subset of the latter type, retrotransposons, have evolved from latent retroviruses that inserted into primordial genomes, and several families contain members that are capable of transposition. We investigated whether retrotransposon sequences are found in salEV cargo. Using the T2T human genome annotation that includes all genomic sequences we found that all 3 classes of retrotransposons were present, including the long interspersed nuclear elements (LINEs), short interspersed nuclear elements (SINEs), and long terminal repeat retrotransposons (LTRs) (FIG. 3A). The total number of retrotransposon transcripts present was highly variable between individual samples. Transcript counts for these elements were relatively high, indicating that they may be targeted to the salEVs. Contrary to what is seen in aged tissue and senescent human cell lines, the centromeric satellite sequences didn't have significant representation in the salEV RNA content. De Cecco et al., Aging Cell, 12(2):247-56 (2013). This result also indicates that retrotransposons may be targeted to the EVs, since the centromeric satellite sequences show the greatest increase in expression in aged tissues and senescent cell lines.

We examined the top 20 represented lncRNA transcripts in each sample and found that 16 lncRNAs were among the top 20 in ≥50% of the samples, indicating a high degree of similarity in lncRNA cargo between individuals. PCBP1-AS1 was the most abundant lncRNA transcript in all but 1 of the samples. This was followed by TEX41, the second most abundant transcript in 13 samples. PVT1 was present in all 16 samples, and was the most abundant transcript in 1 sample, and the third most abundant transcript in 13 samples. LINC00635, MIR99AHG, MUC20-OT1, CCDC26, SNHG14, HULC, MIR4435-2HG, CASC15, SOX2-OT, LINC01206. SNHG5, MIR100HG, and SNH17 comprised the remaining lncRNAs that are among the most abundant across salEV samples. The similarity of lncRNA representation between samples suggests that lncRNA loading into salEVs may not be stochastic and that specific lncRNAs may be targeted to salEVs.

MicroRNAs (miRNAs) are known to be abundant in EVs. To determine which miRNAs are represented in salEVs, we performed Nanostring analysis on the total RNA isolated from 6 of the samples. We found 286 miRNAs that were expressed above background levels. The top 20 miRNA had average counts of 446.7 per sample and all 20 had counts above the low expression cutoff of 100 counts per sample (FIG. 3B). The 2 most abundant miRNAs present were miR-223-3p and miR-155-5p, which are involved in the inflammatory response and are differentially expressed in AD. Liu and Siadat Clin Chim Acta, 578:120506 (2025). In addition, miR-23a-3p, let-7i, miR-16-5p, and miR-320e were included in the top 20 represented miRNAs. These miRNAs are differentially expressed in AD. Kim et al., Exp Gerontol., 136:110954 (2020); Meng et al., J R Soc Interface, 11(92):20131057 (2014); Sorensen et al., Transl Neurodegener, 5:6 (2016); Weinberg et al., Front Neurosci., 9:430 (2015). We used the mirPath software on the Diana Tools website to predict which pathways would be affected by altered expression of the top 20 miRNAs. The most significantly affected pathways contained between 9-181 genes that are predicted to be regulated by between 9-19 of the top 20 miRNAs (FIG. 3C). Several of these pathways are important in cellular function and altered levels of these miRNAs in salEV target cells could affect these cellular processes. These pathways include Hippo signaling, TGF-β signaling, Wnt signaling, FoxO signaling ErbB signaling, axon guidance, and mTOR signaling.

### Comparison to Blood EVs

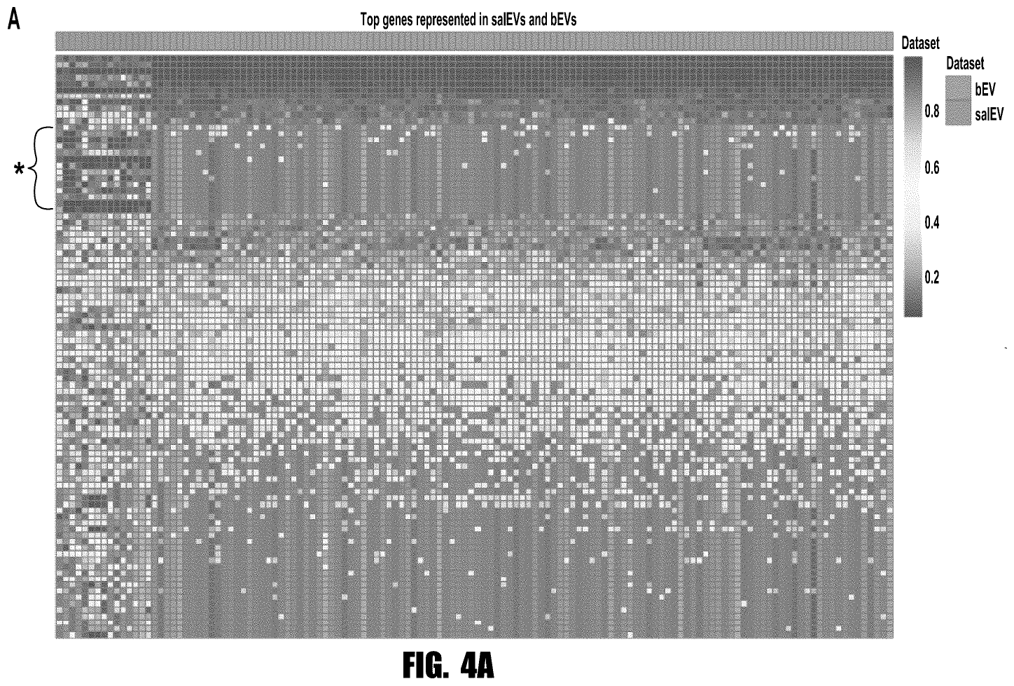

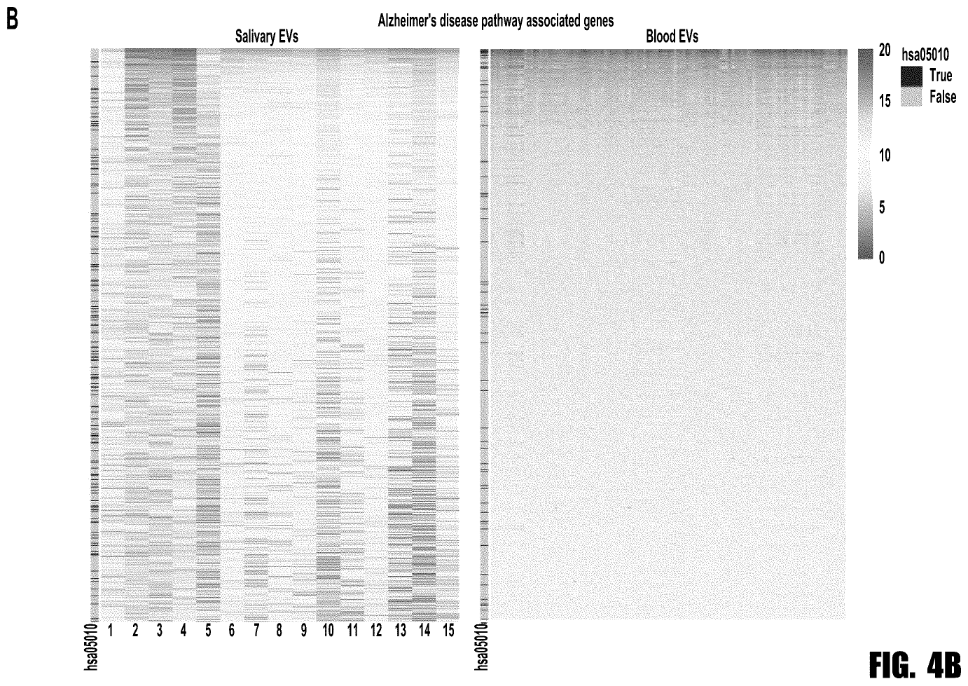

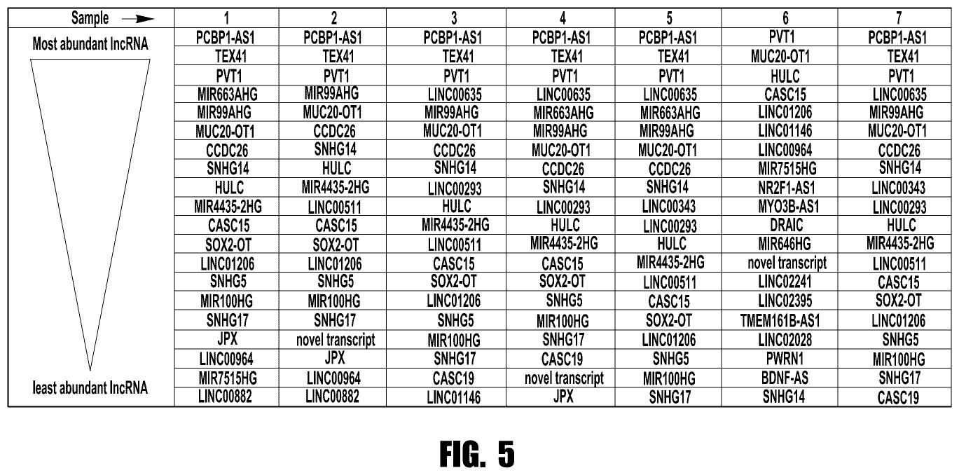

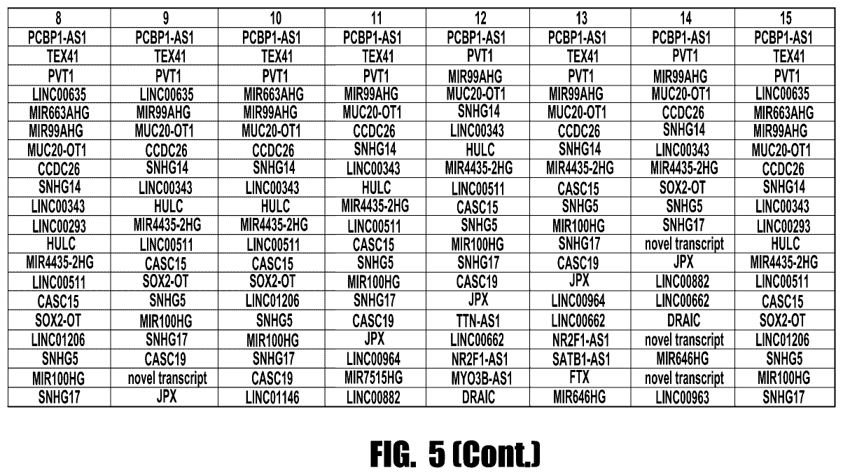

We obtained publicly available RNA-seq data from blood derived EVs (bEVs) from 117 healthy donors. When we contrast the mRNA abundance profiles between salEVs and bEVs, some genes are highly detected in both salEVs and bEVs, but there are a set of genes that are highly present in most salEV samples that are hardly detected in any of the bEV samples (FIG. 4A). As expected, many of these genes are related to salivary secretion. What is encouraging is that many genes associated with neurodegenerative diseases are also detected in salEV, and to a greater extent than their presence in the bEVs. The top 1000 represented transcripts in salEVs include many that are associated with the Alzheimer's disease KEGG pathway (hsa05010) (FIG. 4B). Specifically, among the 390 genes associated with the AD pathway, 128 (32.8%) are among the top 1000 transcripts found in salEVs. In contrast, only 62 (15.9%) of these genes are among the top 1000 transcripts in bEVs. An extended comparison of six neurodegenerative disease-associated KEGG pathways is shown in Table 2, with the corresponding gene abundance heatmaps in FIG. 5. For each neurodegenerative condition, there is higher representation of pathway-associated transcripts in salEVs compared to bEVs. These results show that salEVs may be useful for identifying neurodegeneration-related biomarkers.

TABLE 2
Number of genes associated with neurodegenerative pathways

| | | | | Pathway size - |
|---|---|---|---|---|
| KEGG | Pathway | salEV (%) | bEV (%) | # of genes |
| hsa05010 | Alzheimer disease | 128 (32.8) | 62 (15.9) | 390 |
| hsa05012 | Parkinson disease | 124 (45.9) | 60 (22.2) | 270 |
| hsa05014 | Amyotrophic lateral | 136 (37.2) | 71 (19.4) | 366 |
| | sclerosis | | | |
| hsa05016 | Huntington disease | 119 (38.5) | 53 (17.2) | 309 |
| hsa05020 | Prion disease | 117 (42.2) | 56 (20.2) | 277 |
| hsa05022 | Pathways of | 152 (31.6) | 84 (17.5) | 481 |
| | neurodegeneration - | | | |
| | multiple diseases | | | |

### Discussion

Our group and others have previously shown that the RNA content of salEVs can distinguish between people who have a traumatic brain injury from those who have not. These results led us to investigate whether we can identify transcripts that belong to pathways associated with neurodegeneration in salEVs. However, the RNA content of salEVs has not been well described, and in order to identify changes associated with pathology, we must first establish the RNA content found in cognitively normal individuals without disease. To accomplish this goal, we isolated and characterized EVs from saliva from cognitively normal older individuals above 65 years of age.

The idea that EVs originating in the brain can travel to the saliva is further supported by the fact that certain viruses, such as the rabies virus, show primary pathology in the central nervous system and the viral particles are efficiently targeted to the saliva for transmission. Liu et al., Life Sci., 260:118305 (2022). The rabies virus particle size (90×180 nm) is similar in size to exosomes and microvesicles. The presence of EVs originating from the CNS in the saliva suggests that EVs may behave similarly to the rabies virus and spread centrifugally from the CNS to the salivary glands. The exact mechanism of how the rabies virus and CNS EVs are targeted to the saliva is poorly understood. There are several theories on how EVs originating in the CNS can travel to the saliva. One possibility is through the lymphatic system. The recently discovered glymphatic system, in which cerebrospinal fluid and interstitial fluid travel through the brain parenchyma and drain either back into the CSF or into the meningeal and cervical lymphatic vessels (Eide et al., Sci Rep., 8(1):7194 (2018)), could provide a mechanism of localization to the salivary glands. The cervical lymphatic vessels drain into the cervical lymph nodes, several of which are embedded in the parotid salivary gland or are in close proximity to the submaxillary salivary gland. Ioachim et al., Arch Pathol Lab Med., 112(12):1224-8 (1988). It is possible that EVs released into the interstitial space in the brain parenchyma can be washed into the cervical lymphatic vessels and end up in the cervical lymph nodes present near the salivary glands and pass into the saliva. It is also possible that the EVs can travel to the salivary gland via the cranial nerves that innervate these glands. EVs have also been shown to cross the blood brain barrier and enter the circulatory system, which could result in their accumulation in the saliva by transcytosis from the blood. Banks et al., Int J Mol Sci., 21(12) (2020). The exact mechanism that results in accumulation of CNS EVs in the saliva is an area for future investigations.

It was previously reported that a large proportion of extracellular RNA (exRNA) found in saliva is of microbial origin. Kaczor-Urbanowicz et al., Bioinformatics, 34(1):1-8 (2018). However, this report included all exRNA, not just what is present in salEVs. Kaczor-Urbanowicz and colleagues found that approximately 17% of reads mapped to human genome, we found a greater number of reads mapped to the human genome, ˜48.4% mapped uniquely or multi-mapped to the T2T human genome. The isolation of the salEVs resulted in a reduction in RNA originating from bacteria and other species present in the oral cavity, likely due to the contaminating RNA being either free floating or present in complexes that were removed with the lower speed centrifugation we used prior to the ultracentrifugation to isolate the EVs. By isolating the EVs from saliva we are enriching for RNA of human origin, allowing us to identify specific transcripts that may indicate a pathology or disease.

We analyzed the RNA content of EVs isolated from saliva to determine the types of transcripts present in this vesicle type. We found that a large proportion of the RNA transcripts mapped to coding genes. The 2023 release of NextProt reveals that the human genome contains 20,389 protein coding genes. We found an average of 12,213 protein coding genes represented in the transcriptomic data from salEVs, indicating that approximately 60% of the coding genes present in the genome were represented in salEVs. This broad presence of protein-coding genes in salEVs suggests EVs could be a valuable source for identifying biomarkers linked to different diseases or conditions.

Despite the large percentage of represented genes, analysis of the regulatory regions of these genes indicates that many of the transcripts may be targeted to salEVs rather than stochastically packaged into the EV. Analysis of transcription factor binding motifs in the promoter regions of transcripts found in salEVs revealed several binding motifs that were highly represented in the data (FIG. 2D). AP-2ε was the most abundant transcription factor binding motif found in transcripts isolated from salEVS. AP-2ε has been shown to play a role in development and has been studied in the context of cancer where it may play a role in regulating apoptosis (Hoshi et al., Oncol Rep., 37(4):2459-64 (2017)), however its role in normal adult cellular processes has not been examined. Our results indicate that AP-2ε is active in the cells of origin in older adults and AP-2ε may influence the cellular physiology of target cells through the transfer of AP-2ε regulated genes.

Binding motifs for three members of the bHLH TF family were also identified as abundant in the regulatory regions of represented protein coding genes found in salEVs. These include HEYL, HES4, and TCFL5. These bHLH TFs are expressed during development and have been shown to regulate multiple developmental pathways, including the differentiation of neural progenitor cells into neurons. Jalali et al., J Neurosci Res., 89(3):299-309 (2011). These TFs have been shown to modulate transcription of their target genes by mostly downregulating their target transcripts rather than creating an on/off process. Therefore, the fact that we see transcripts containing the bHLH TF binding sites is not surprising, since the salEVs were collected from older adults where developmental pathways would not be expected to be active. These results suggest that these TFs are not being actively expressed in the cells of origin of the salEVs since these TFs downregulate expression of their gene targets, and we see these transcripts enriched in salEVs in older adults.

Our analysis showed that salEVs contain large numbers of lncRNAs, and that the lncRNAs present were similar across different samples from different individuals, suggesting that these lncRNAs may be preferentially packaged into the EVs. The most abundant lncRNAs found in human cells, such as MALATI and NEAT1, are not highly represented in salEVs, further supporting the idea that the lncRNAs present may be selectively targeted to salEVs. There are currently more than 20,000 lncRNAs annotated in the human genome and efforts are underway to expand on that number. Despite the large number of annotated lncRNAs, very little is known about the function of the majority of the lncRNAs. The most abundant lncRNAs in most of the salEV samples were PCBP1-AS1, TEX41, and PVT1. PCBP1-AS1, has been studied in the context of cancer, where it either promotes or inhibits cancer progression depending on the cancer type. Wu et al., Medicine (Baltimore), 102(43):e35631 (2023). Interestingly, human tissue expression studies show that PCBP1-AS1 has higher expression in the brain than in many other tissues (Fagerberg et al., Mol Cell Proteomics, 13(2):397-406 (2014)), however the role it plays in normal brain function and neurodegeneration has not been studied. TEX41 has also been studied in the context of cancer where it may play a role in regulating autophagy by increasing Runx2 levels, and may control cancer cell proliferation and migration by regulating miR153-3p levels. It may also play a role in calcific aortic stenosis. The role of TEX41 in the healthy brain and in neurodegeneration has not been studied. PVT1, on the other hand, has been shown to be upregulated in the brain in several pathological conditions, including ischemic stroke and glioblastoma. Lu et al., Life Sci., 260:118305 (2020); Zhang et al., Experimental and therapeutic medicine, 17(2):1337-45 (2019). It will be interesting to determine if levels of these lncRNAs correlate with neurodegenerative changes in the brain and if they can be used as biomarkers of these conditions.

miRNAs are abundant in EVs from all cell types, and we examined the miRNAs present in salEVs. We found 6 miRNAs present in salEVs that are differentially expressed in inflammation and AD (FIG. 2B), suggesting that levels of these miRNAs in salEVs may be used as biomarkers for these conditions. We examined the pathways predicted to be affected by the top 20 miRNAs found in salEVs (FIG. 3C). Perturbation of these pathways has been shown to occur in several neurodegenerative conditions. Hippo signaling is known to regulate neural stem cell differentiation, including neuronal differentiation and glial differentiation. TGF-β signaling is involved in anti-inflammatory responses and has neuroprotective effects, and TGF-β deficiency promotes Δβ deposition and neuronal loss in a mouse model of AD Tesseur et al., The Journal of clinical investigation, 116(11):3060-9 (2006). Wnt signaling is dysfunctional in AD, leading to neuropathological conditions. Arredondo et al., Front Cell Dev Biol., 8:860 (2020). FoxO signaling is involved in the downregulation of IGF1 in AD. Kang et al., Oxid Med Cell Longev., 2022:8169981 (2022). mTor is involved in activation of NF-ϰB that triggers the expression of chemokines and inflammatory cytokines. Singh et al., Ageing Res Rev. 2024; 98:102321 (2024). These results show that miRNAs that affect pathways that may contribute to neurodegeneration can be found in salEVs, and investigating the miRNA content of salEVs may reveal risk of neurodegenerative changes before the onset of cognitive impairment.

We found a significant representation of transcripts that originated from repetitive regions of the genome. Specifically, there were large numbers of transcripts from retrotransposons. These sequences arose from retroviruses that inserted into a primordial genome. Most transposable elements in the genome are inactive due to the accumulation of mutations that have occurred over evolutionary time. However, a few transcripts are intact and encode the machinery necessary for insertion into novel locations in the target cell genome, leading to potential mutations, genomic rearrangements, and genomic instability. In addition, retrotransposon expression has been linked to activation of the innate immune response resulting in inflammation. Interestingly, retrotransposons have been shown to increase in expression with age, and we see significant representation of these sequences in salEVs isolated from individuals over the age of 65. This age-associated increase is further augmented in neurodegenerative conditions. Frost and Dubnau Annu Rev Neurosci., 47(1):123-43 (2024). For example, the long terminal repeat retrotransposons are elevated in TDP43 pathologies (Douville et al., Ann Neurol., 69(1):141-51 (2011)); and tauopathies, including Alzheimer's disease, are thought to drive neurodegeneration by activating retrotransposons. In addition, inhibitors of the long interspersed nuclear elements and short interspersed nuclear element retrotransposons protect against neurodegeneration and cognitive decline in multiple aging model systems. Wahl et al., Aging Cell, 22(5):e13798 (2023). We found a significant number of retrotransposon transcripts are present in salEV cargo in individuals over the age of 65, which leads to the question of whether retrotransposon transcripts are spread to target cells through EVs resulting in the spread of age-associated inflammation and other degenerative processes.

Recently, bEVs have been investigated for potential biomarkers of disease, including cancer and neurodegenerative conditions (Bazan Russo et al., Future Oncol., 1-19 (2025); Torrini et al., Trends Pharmacol Sci., 46(5):468-79 (2025). In this study we found greater representation of neurodegeneration-related transcripts in salEVs (FIG. 4), indicating that salEVs may be a better source for identifying biomarkers of neurodegenerative diseases than bEVs. For 6 different neurodegenerative disease KEGG pathways, salEVs had approximately twice the number of pathway-associated transcripts than what was found in bEVs (Table 2). Due to the greater representation of neurodegeneration-related transcripts, and the presence of other transcripts such as retrotransposons that are known to be associated with these conditions, salEVs may serve as an effective, non-invasive alternative to bEVs.

### CONCLUSIONS

The data presented in the manuscript show that the RNA cargo of salEVs contain transcripts that are associated with neuronal function. By analyzing these transcripts, we may be able to determine if an individual is at a heightened risk for developing a neurodegenerative condition. Saliva can be collected in a non-invasive manner at a point-of-care facility without any specialized training, increasing access to screening for these debilitating conditions. Future studies will focus on identifying transcripts that indicate if someone may be a candidate for more in-depth testing and treatments to slow the progression of these devastating neurodegenerative diseases.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of identifying subjects having an increased risk of having or developing Alzheimer's disease (AD) or an Alzheimer's disease-related disorder (ADRD), comprising:
obtaining a saliva sample from the subject;
isolating extracellular vesicles from the saliva sample;
determining the level of a category of RNA present in the extracellular vesicles;
comparing the level of the category of RNA to a control level; and
determining that the subject has an increased risk of having or developing AD or ADRD if the category of RNA level is altered relative to that of the control level.

2. The method of claim 1, wherein the category of RNA is up-regulated in subjects having an increased risk of having or developing AD or an ARDR.

3. The method of claim 1, wherein the category of RNA is down-regulated in subjects having an increased risk of having or developing AD or an ARDR.

4. The method of claim 1, wherein the method is used to identify a subject having an increased risk of having or developing Alzheimer's disease.

5. The method of claim 1, wherein the method comprises determining the level of a plurality of microRNA.

6. The method of claim 5, wherein the microRNA is selected from the group consisting of hsa-miR-498-3p, hsa-miR-614, hsa-miR-107, hsa-miR1249-5p, and hsa-miR-519-3p.

7. The method of claim 1, wherein the method comprises determining the level of a plurality of mRNA.

8. The method of claim 1, wherein the method comprises determining the level of a plurality of lncRNA.

9. The method of claim 1, wherein the method comprises determining the level of a plurality of retrotransposons.

10. The method of claim 9, wherein the retrotransposons comprise LINE and SINE families of retrotransposons.

11. The method of claim 1, wherein the RNA is enriched for AP-2ε, HEYL, HES4, and TCFL5 transcription factor binding sites.

12. The method of claim 1, wherein the step of determining the level of a category of RNA present in the extracellular vesicles is carried out using a point-of-care device.

13. The method of claim 1, further comprising treating the subject identified as having an increased risk of having or developing AD or ARDR.

14. A method of evaluating treatment of Alzheimer's disease (AD) or an Alzheimer's disease-related disorder (ARDR) in a subject receiving treatment for, comprising:
obtaining a saliva sample from the subject;
isolating extracellular vesicles from the saliva sample;
determining the level of a category of RNA present in the extracellular vesicles;
comparing the level of the category of RNA to a control level corresponding to the biomarker level before treatment; and
determining if the treatment of the AD or ARDR is effective if the category of RNA level is altered relative to that of the control level.

15. The method of claim 14, wherein the category of RNA is up-regulated in subjects where treatment of the AD or ARDR is effective.

16. The method of claim 14 wherein the category of RNA is down-regulated in subjects where treatment of the AD or ARDR is effective.

17. The method of claim 14, wherein the method is used to identify a subject being treated for Alzheimer's disease.

18. The method of claim 14, wherein the method comprises determining the level of a microRNA.

19. The method of claim 18 wherein the microRNA is selected from the group consisting of hsa-miR-498-3p, hsa-miR-614, hsa-miR-107, hsa-miR1249-5p, and hsa-miR-519-3p.

20. The method of claim 14, wherein the method comprises determining the level of a plurality of mRNA.

21. The method of claim 14, wherein the method comprises determining the level of a plurality of lncRNA.

22. The method of claim 14, wherein the method comprises determining the level of a plurality of retrotransposons.

23. The method of claim 22, wherein the retrotransposons comprise LINE and SINE families of retrotransposons.

24. The method of claim 14, wherein the RNA is enriched for AP-2ε, HEYL, HES4, and TCFL5 transcription factor binding sites.

25. The method of claim 14, wherein the step of determining the level of the category of RNA present in the extracellular vesicles is carried out using a point-of-care device.


SEQUENCE LISTING (5 sequences)

SEQ ID NO: 1 - RNA, 23 nt, Homo sapiens
  source 1..23: mol_type = other RNA
aaagcacctccagagcttgaagc

SEQ ID NO: 2 - RNA, 23 nt, Homo sapiens
  source 1..23: mol_type = other RNA
gaacgcctgttcttgccaggtgg

SEQ ID NO: 3 - RNA, 23 nt, Homo sapiens
  source 1..23: mol_type = other RNA
agcagcattgtacagggctatca

SEQ ID NO: 4 - RNA, 24 nt, Homo sapiens
  source 1..24: mol_type = other RNA
aggagggaggagatgggccaagtt

SEQ ID NO: 5 - RNA, 22 nt, Homo sapiens
  source 1..22: mol_type = other RNA
caaagtgcctccctttagagtg